(19) United States
(12) Patent Application Publication
(10) Pub. No.: US 2006/0147548 A1
(43) Pub. Date: Jul. 6, 2006

(54) Therapeutic delivery of carbon monoxide

(76) Inventors: Roberto Angelo Motterlini, Middlesex (GB); Brian Ernest Mann, Sheffield (GB)

Correspondence Address: NIXON & VANDERHYE, PC, 901 NORTH GLEBE ROAD, 11TH FLOOR, ARLINGTON, VA, 22203, US

(21) Appl. No.: 10/535,226

(22) PCT Filed: Nov. 20, 2003
(86) PCT No.: PCT/GB03/05061

§ 371 Date: Dec. 16, 2005

(30) Foreign Application Priority Data
Nov. 20, 2002 (GB) 0227135.1

(51) Int. Cl. A61K 33/24; A61K 31/28

(52) U.S. Cl. 424/617; 514/492

(57) ABSTRACT

Metal carbonyls are used in combination with at least one guanylate cyclase stimulant/stabilizer to deliver CO having biological activity, for example vasodilatation and inhibition of platelet aggregation. The two components may be administered simultaneously or sequentially. A particular described combination is tricarbonylchloro(glycinato)ruthenium(II) and the drug YC-1.

### FIELD OF THE INVENTION

The present invention relates to pharmaceutical preparations, particularly preparations for therapeutic delivery of carbon monoxide to humans and other mammals, to methods of delivery of therapeutic agents and to kits for this purpose.

### BACKGROUND OF THE INVENTION

The vasodilatory effects of nitric oxide (NO) and carbon monoxide (CO) gases have been known for some time (3). The L-arginine/NO synthase pathway present in the vascular endothelium plays a fundamental role in the control of vessel relaxation and arterial blood pressure in mammals (4). Increased generation of carbon monoxide (CO) following activation of the heme oxygenase-1 enzyme in the vascular tissue also results in suppression of acute hypertension in vivo (6) and prevention of vasoconstriction ex vivo (7).

Most recently, it has been reported that a series of transition metal carbonyls can be utilized as CO-releasing molecules (CO-RMs) in biological systems to elicit vasorelaxation and prevent increases in blood pressure (5).

Vascular relaxation by NO and CO appears to involve an increase in intracellular cyclic 3′,5′-guanosine monophosphate (cGMP) levels through activation of a soluble heme-dependent guanylate cyclase (sGC) (3; 6; 7). However, it is known that CO is a poor stimulator of sGC in in vitro studies when compared to NO; the enzymatic activity of purified guanylate cyclase is increased 130-fold and 4.4-fold by its interaction with NO and CO, respectively (8).

Interestingly, data from the literature reveal that the catalytic rate of sGC can be substantially improved by the benzyl-indazole derivative 3-(5′-hydroxymethyl-2′- furyl)-1-benzyl-indazole (YC-1). The mechanism underlying YC-1 action may be the stabilization of guanylate cyclase in its active conformation. It has also been suggested that YC-1 may stimulate production of guanylate cyclase.

W002/092075, published 21 Nov. 2002 and originating from work of the present inventors, discloses various metal carbonyl compounds that can be used in the delivery of carbon monoxide to body cells and tissue. Some of the metal carbonyl compounds disclosed therein typically included a ligand other than CO and can be employed in the present invention. There is a statement that YC-1 may be used as a ligand.

### SUMMARY OF THE INVENTION

An object of the present invention is to provide method of achieving improved therapeutic effects by delivery of carbon monoxide to the human or other mammal body.

As exemplified by the experimental data detailed below, the present inventors have found that metal carbonyl compounds can be used in combination with a guanylate cyclase stimulant or stabilizer so as to provide an improved physiological effect.

Accordingly, in a first aspect, the present invention provides a pharmaceutical preparation, comprising a metal carbonyl compound or pharmaceutically acceptable salt thereof, a guanylate cyclase stimulant or stabilizer and at least one pharmaceutically acceptable carrier. Typically the metal carbonyl makes available CO suitable for physiological effect, for delivery of carbon monoxide to a physiological target.

The preparation may contain the metal carbonyl and guanylate cyclase stimulant/stabilizer in a single composition, or the two components may be formulated separately for simultaneous or sequential administration.

In a second aspect, the present invention provides a method of a therapeutic agent to a mammal comprising the step of administering a pharmaceutical preparation according to the first aspect.

In a third aspect, the present invention provides a method of introducing therapeutic agent to a mammal, comprising:

a) administering a metal carbonyl; and

b) administering a guanylate cyclase stimulant or stabiliser.

The metal carbonyl and guanylate cyclase stimulant/stabilizer may be administered simultaneously either in a single composition or in two separate compositions. The metal carbonyl and stimulant/stabilizer may be administered sequentially. Preferably, the stabilizer/stimulant is administered first followed by the metal carbonyl but this order may be reversed.

In a fourth aspect, the invention provides a kit comprising a) a metal carbonyl compound and b) a guanylate cyclase stimulant/stabilizer.

The two components may be for administration simultaneously or sequentially.

While the invention is primarily here discussed as involving the delivery of carbon monoxide to a physiological target wherein the metal carbonyl makes CO available for physiological effect, it is not excluded that a different mechanism is involved, such as that the metal carbonyl acts directly without release of CO.

The various aspects of this invention are useful for treating a variety of body tissues in a living mammal. Additionally, isolated organs, e.g. extracorporeal organs or in situ organs isolated from the blood supply, can be treated. The organ may be, for example, a circulatory organ, respiratory organ, urinary organ, digestive organ, reproductive organ, neurological organ, muscle or skin flap or an artificial organ containing viable cells. In particular, the organ may be a heart, lung, kidney or liver. However, the body tissue which is treatable are not limited and may be any human or mammal body tissue whether extracorporeal or in situ in the animal body.

The various aspects of the present invention are used to provide a physiological effect, e.g. for stimulating neurotransmission or vasodilation, or for treatment of any of hypertension, such as acute, pulmonary and chronic hypertension, radiation damage, endotoxic shock, inflammation, inflammatory-related diseases such as asthma and rheumatoid arthritis, hyperoxia-induced injury, apoptosis, cancer, transplant rejection, arteriosclerosis, post-ischemic organ damage, myocardial infarction, angina, haemorrhagic shock, sepsis, penile erectile dysfunction, adult respiratory distress syndrome and inhibition of platelet aggregation.

The various aspects can also be used for perfusion, of a viable mammalian organ extracorporeally, e.g. during storage and/or transport of an organ for transplant surgery or treatment of an organ which is in the body but is temporarily isolated from the bloodstream, e.g. during surgery. For this purpose, the metal carbonyl is in dissolved form, preferably in an aqueous solution.

In the various aspects of the present invention, preferably, the metal carbonyl makes CO available by at least one of the following means:

1) CO derived by dissociation of the metal carbonyl is present in the composition in dissolved form;

2) on contact with a solvent or ligand the metal carbonyl releases CO;

3) on contact with a tissue, organ or cell the metal carbonyl releases CO;

4) on irradiation the metal carbonyl releases CO.

The most preferred metal carbonyls are water soluble metal carbonyls.

Certain metal carbonyl compounds are capable of releasing CO on contact with a suitable solvent. When the metal carbonyl component is to be administered in liquid form, this solvent may form a part of the component. Thus, the pharmaceutical preparation contains CO derived from the metal carbonyl in dissolved form. The conditions under which the carbonyl compound is dissolved in the solvent during preparation of the metal carbonyl component may be controlled such that the CO thus released is retained in solution. This may be facilitated where an equilibrium exists between the dissociated components and the undissociated carbonyl.

The dissociated components of the parent carbonyl may themselves be metal carbonyl complexes capable of releasing further CO. For example, when [Ru(CO)₃Cl₂]₂ is dissolved in DMSO, CO is liberated into solution, and a mixture of tri-carbonyl and di-carbonyl complexes is formed, and these themselves may be capable of releasing further CO.

Alternatively, the metal carbonyl component may not itself contain dissolved CO, but may be prepared such as to release CO on contact with a suitable solvent or medium. For example, the composition may contain a metal carbonyl compound capable of releasing CO on contact with water, e.g. on contact with an aqueous physiological fluid, such as blood or lymph. The metal carbonyl compound may also release CO on contact with perfluorocarbon type blood substitute fluids or on contact with cardioplegic fluid.

Alternatively, the pharmaceutical composition may be intended to be dissolved in water prior to administration. Such metal carbonyl components may be prepared in solution or in solid form, such as in tablet form. If they are in solution form, they will typically be prepared in a solvent which does not support dissociation of the metal carbonyl compound, such that release of CO takes place only on contact with the appropriate substance.

Alternatively or additionally, release of CO from the carbonyl can be stimulated by reaction with a ligand in solution which for example replaces one of the ligands of the complex leading to loss of CO from the complex. The ligand may be one containing sulphur or nitrogen. Some metal carbonyls may release CO on contact with biological ligands such as glutathione or histidine.

As another alternative, the metal carbonyl component may contain a metal carbonyl compound which releases CO on contact with a tissue, organ or cell. It is known that certain metal carbonyl compounds do not release CO to solution but are nevertheless capable of releasing CO to physiological cellular materials or tissues, such as vascular endothelium. For example, [Fe(SPh)₂(2,2′-bipyridine) (CO)₂] does not release CO to myoglobin in solution, but is nevertheless capable of promoting dilatation of pre-contracted aortic rings. Without wishing to be limited by any particular theory, it is thought that CO may be released from such compounds as a result of an oxidation-reduction reaction, mediated by cellular components such as cytochromes.

However the invention is not limited to a redox reaction as a mechanism for CO release, since loss of at least a first CO molecule from the complex may occur without redox.

As yet another alternative, the metal carbonyl component may contain a metal carbonyl compound which releases CO on irradiation. The compound may be irradiated prior to administration, for example to produce a solution of dissolved CO, or may be irradiated in situ after administration. It is contemplated that such compositions may be used to provide controlled, localised release of CO. For example a pharmaceutical composition of this type may be administered during surgery, and CO released specifically at a site in need thereof, e.g. to induce vasodilation, by localised irradiation by means of a laser or other radiant energy source, such as UV rays.

Typically the metal carbonyl components of the present invention release CO such as to make it available to a therapeutic target in dissolved form. However, in some circumstances CO may be released from a metal carbonyl directly to a non-solvent acceptor molecule.

Typically the metal carbonyl compound comprises a complex of a transition metal, preferably a transition metal from group 6 to 10 (in this specification the groups of the periodic table are numbered according to the IUPAC system from 1 to 18). The number of carbonyl ligands is not limited, provided at least one carbonyl ligand is present. The preferred metals are transition metals of lower molecular weight, in particular Fe, Ru, Mn, Co, Ni, Mo and Rh. Two other metals which may be used are Pd and Pt. In the metal carbonyl complexes used in the invention, the metal is typically in a low oxidation state, i.e. O, I or II. For the metals preferred, the oxidation states are typically not higher than Fe^{II}, Ru^{II}, Mn^{I}, Co^{II}, preferably Co^{I}, Rh^{III} preferably Rh^{I}, Ni^{II}, Mo^{II}. The metal is preferably not a radionuclide. Fe is one particularly suitable metal, since Fe is present in quantity in mammals.

The metal carbonyl compounds may be regarded as complexes, because they comprise CO groups coordinated to a metal centre. However the metal may be bonded to other groups by other than coordination bonds, e.g. by ionic or covalent bonds. Thus groups other than CO which form part of the metal carbonyl compound need not strictly be “ligands” in the sense of being coordinated to a metal centre via a lone electron pair, but will be referred to herein as “ligands” for ease of reference.

Thus, the ligands to the metal may all be carbonyl ligands, as e.g. in :(Mn₂(CO)₁₀). Alternatively, the carbonyl compound may comprise at least one modulatory ligand. By this is meant a ligand which is not CO, but which modulates a particular property of the complex, such as the tendency to release CO, solubility, hydrophobicity, stability, electrochemical potential, etc. Thus suitable choices of ligand may be made in order to modulate the behaviour of the compound. For example it may be desirable to modulate the solubility of the compound in organic and/or aqueous solvents, its ability to cross cell membranes, its rate of release of CO on contact with a particular solvent or cell type, or on irradiation, etc.

Such ligands are typically neutral or anionic ligands, such as halide, or derived from Lewis bases and having N, P, O, S or C as the coordinating atom(s). Preferred coordinating atoms are N, O and S. Examples include, but are not limited to, sulfoxides such as dimethylsulfoxide, natural and synthetic amino acids and their salts for example, glycine, cysteine, and proline, amines such as NEt₃ and H₂NCH₂CH₂NH₂, aromatic bases and their analogues, for example, bi-2,2′-pyridyl, indole, pyrimidine and cytidine, pyrroles such as biliverdin and bilirubin, thiols and thiolates such as EtSH and PhSH, chloride, bromide and iodide, carboxylates such as formate, acetate, and oxalate, ethers such as Et₂O and tetrahydrofuran, alcohols such as EtOH, and nitriles such as MeCN. Particularly preferred are coordinating ligands, such as amino acids, which render the carbonyl complex stable in aqueous solution. Other possible ligands are conjugated carbon groups, such as dienes. One class of ligands which can provide metal carbonyl compounds of use in this invention is cyclopentadienyl (C₅H₅) and substituted cyclopentadienyl. The substituent group in substituted cyclopentadienyl may be for example an alkanol, an ether or an ester, e.g. -(CH₂)ₙOH where n is 1 to 4, particularly —CH₂OH, -(CH₂)ₙOR where n is 1 to 4 and R is hydrocarbon preferably alkyl of 1 to 4 carbon atoms and -(CH₂),ₙOOCR where n is 1 to 4 and R is hydrocarbon preferably alkyl of 1 to 4 carbon atoms. The preferred metal in such a cyclopentadienyl or substituted cyclopentadienyl carbonyl complex is Fe. Preferably the cyclopentadienyl carbonyl complex is cationic, being associated with an anion such as chloride.

Thus the properties of pharmaceutical compositions of the present invention may be tailored as required by appropriate choice of metal centres and number and type of associated ligands in the metal carbonyl compound.

The metal carbonyl compound may further comprise a targeting moiety, to facilitate release of CO at an appropriate site. The targeting moiety is typically capable of binding a receptor on a particular target cell surface, in order to promote release of CO at the required site. The targeting moiety may be a part of a modulating ligand capable of binding to a receptor found on the surface of the target cells, or may be derived from another molecule, such as an antibody directed against a particular receptor, joined to the complex by a suitable linker.

The present invention also includes as the metal carbonyl component a compound of the formula M(CO)ₓA_{y} where x is at least one, y is at least one, M is a metal, A is an atom or group bonded to M by an ionic, covalent or coordination bond but is not CO, and, in the case where y>1, each A may be the same or different, or a pharmaceutically acceptable salt of such a compound. Typically, M is a transition metal, particularly of groups 6 to 10, and A may be selected from neutral or anionic ligands, such as halide, or derived from Lewis bases and having N, P, O, S or C as the coordinating atom(s). Mono-, bi- or polydentate ligands may be used. More details of preferred metals and ligands are given above. The molecular weight of this compound is preferably less than 1000, e.g. not more than 822. Some CO-releasing carbonyls and their release properties are given in FIGS. 3A-3F.

The carbonyl complex should be pharmaceutically acceptable, in particular non-toxic or of acceptable toxicity at the dosage levels envisaged.

The metal carbonyl component may be a compound of the formula

M(CO)ₓ A_{y}B_{z} where

M is Fe, Co or Ru,

x is at least one,

y is at least one,

z is zero or at least one,

each A is a ligand other than CO and is monodentate or polydentate with respect to M and is selected from the amino acids
alanine
arginine
asparagine
aspartic acid
cysteine
glutamic acid
glutamine
glycine
histidine
isoleucine
leucine
lysine
methionine
phenylalanine
proline
serine
threonine
tryptophan
tyrosine
valine

[O(CH₂COO)₂^{]2-} and

[NH(CH₂COO)₂^{]2-, and}

B is optional and is a ligand other than CO.

x is preferably 3, y is preferably 1 and z is preferably 1.

The term amino acid here used includes the species obtained by loss of the acidic hydrogen, such as glycinato.

B_{z} represents one or more optional other ligands. There are no particular limitations on B, and ligands such as halides, e.g. chloride, bromide, iodide, and carboxylates, e.g. acetate may be used.

M is selected from Fe, Ru and Co. These metals are preferably in low oxidation states, as described above.

Use of the known iron compounds [Fe(SPh)₂(2,2′-bipyridine) (CO)₂] and [Fe(SPh)₂(NH₂CH₂CH₂NH₂) (CO)₂] is also envisaged in this invention.

The guanylate cyclase stabilizer/stimulant compound may be any compound which stimulates production of guanylate cyclase or which stabilizes guanylate cyclase, in particular the active form of guanylate cyclase. A single compound can be used or a combination of compounds can be used either for simultaneous or sequential administration, i.e. the various aspects include/use at least one guanylate cyclase stimulant/stabilizer.

Examples include 3-(5′-hydroxymethyl-2′-furyl)-1-benzyl-indazole (YC-1), 4 pyrimidinamine-5-cyclopropyl-2-[1-[(2-fluorophenyl)methyl]-1H-pyrazolo[3,4-b]pyridin-3-yl] (BAY 41-2272), BAY 50-6038 (ortho-PAL), BAY 51-9491 (meta PAL), and BAY 50-8364 (para PAL). The structures of ortho-, meta- and para- PAL are shown in FIG. 2. These compounds have been found to bind to an activation site on the guanylate cyclase (9) and any other compounds that similarly bind to the site may be useful as the guanylate cyclase stabilizer/ stimulant. Also useful are NO donors and 1-benzyl-3-(3¹-ethoxycarbonyl)phenyl-indazole, 1-benzyl-3-(3¹-hydroxymethyl)phenyl-indazole, 1-benzyl-3- (5¹-diethylaminomethyl) -furyl-indazole, 1-benzyl-3-(5¹-methoxymethyl)furyl-indazole, 1-benzyl-3-(5¹-hydroxymethyl)furyl-6-methyl-indazole, 1-benzyl-3-(5¹-hydroxymethyl) -furyl-indazol-benzyl-3-(5¹-hydroxymethyl)-furyl-indazole, 1-benzyl-3-(5¹-hydroxymethyl)-furyl-6-fluoro-indazole, 1-benzyl-3-(5¹-hydroxymethyl)-furyl-6-methoxy-indazole, and 1-benzyl-3-(5¹-hydroxymethyl)-furyl-5,6-methylenedioxoindazole or pharmaceutically acceptable salts thereof.

The metal carbonyl component and/or guanylate cyclase stabilizer/stimulant component typically comprise a pharmaceutically acceptable excipient, carrier, buffer, stabiliser or other materials well known to those skilled in the art. Such materials should be non-toxic and should not interfere unduly with the efficacy of the active ingredient. The precise nature of the carrier or other material may depend on the route of administration, e.g. oral, intravenous, subcutaneous, nasal, intramuscular, intraperitoneal, or suppository routes.

Components/preparations for oral administration may be in tablet, capsule, powder or liquid form. A tablet may include a solid carrier such as gelatin or an adjuvant or a slow-release polymer. Liquid compositions/preparations generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Pharmaceutically acceptable amounts of other solvents may also be included, in particular where they are required for dissolving the particular metal carbonyl compound contained in the composition. The composition may further comprise pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); preservatives (e.g. methyl or propyl-p-hydroxybenzoates or sorbic acid); and energy sources (e.g. carbohydrates such as glucose, fats such as palmitate or amino acid).

For intravenous, cutaneous or subcutaneous injection, or injection at the site of affliction, the active ingredient will typically be in the form of a parenterally acceptable solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. Delivery systems for needle-free injection are also known, and compositions for use with such systems may be prepared accordingly.

Administration is preferably in a prophylactically effective amount or a therapeutically effective amount (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

When formulating compositions/preparations according to the present invention, the toxicity of the active ingredient, stimulant/stabilizer and/or the solvent must be considered. The balance between medical benefit and toxicity should be taken into account. The dosages and formulations will typically be determined so that the medical benefit provided outweighs any risks due to the toxicity of the constituents. Examples include St Thomas Hospital solutions, Euro-Collins solutions, University of Wisconsin solutions, Celsior solutions, Ringer Lactate solutions, Bretschneider solutions and perflurocarbons.

The metal carbonyl compound and the stimulant/stabilizer can be formulated into a single composition that can be in any physical form. In this case, the components will be administered simultaneously. Alternatively, the components can be formulated into two compositions which can be administered simultaneously or sequentially.

Throughout this application, references to medical treatment are intended to include both human and veterinary treatment, and references to pharmaceutical compositions are accordingly intended to encompass compositions for use in human or veterinary treatment.

### INTRODUCTION OF THE DRAWINGS

Experimental data illustrating the present invention will now be described by reference to the accompanying figures, in which:

FIG. 1A shows vasodilatory effects of CORM-3 alone and in combination with YC-1 obtained in Example 1;

FIG. 1B shows percentage relaxation obtained in Example 1;

FIG. 2 shows structures of ortho-, meta- and para- PAL; and

FIGS. 3A to F show carbon monoxide releasing molecules;

FIGS. 4A and 4B show absorbance data obtained in Example 2;

FIG. 5 shows relaxation date of Example 3;

FIGS. 6A and 6B show data of Example 4A;

FIGS. 7A, 7B, 7C and 7D show data of Example 4B;

FIGS. 8A and 8B show data of Example 4C; and

FIG. 9 shows data of Example 5.

### EMBODIMENTS OF THE INVENTION AND EXAMPLES

Stock solutions of CORM-3 (100 mM) were prepared by solubilizing the compound in distilled water prior to the experiment. Tricarbonyldichloro ruthenium (II) dimer ([Ru(CO)₃Cl₂]₂) , 3-(5′-hydroxymethyl-2′-furyl)-1-benzyl-indazole (YC-1) and all other reagents were purchased from Sigma-Aldrich (Poole, Dorset).

All data are expressed as mean ± s.e.m. Differences between the groups analysed were assessed by the Student's two-tailed t-test, and an analysis of variance (ANOVA) was performed where more than two treatments were compared. Results were considered statistically significant at P<0.05.

In the Examples below, “mM” and “μM” signify concentrations (millimolar and micromolar respectively).

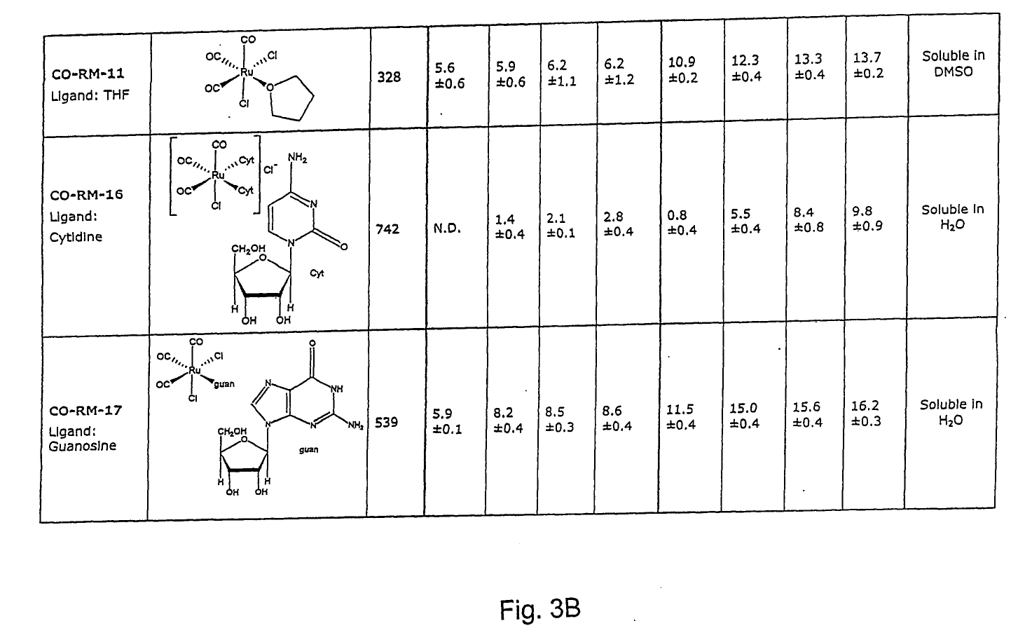

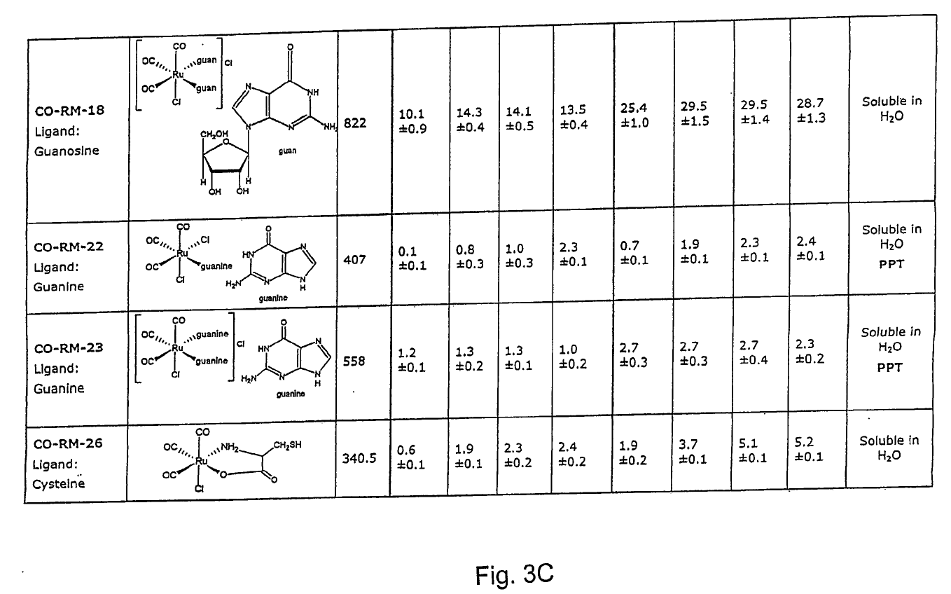

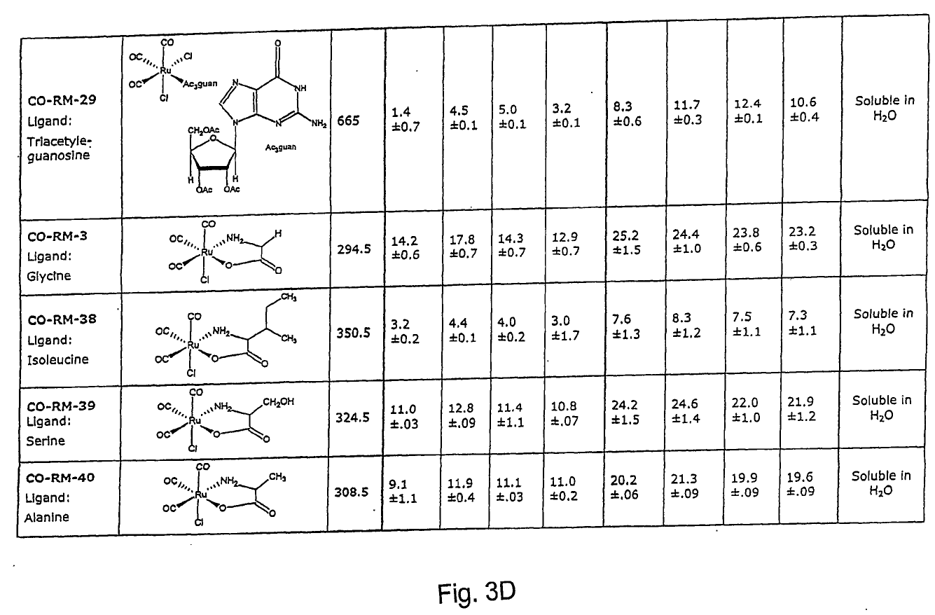

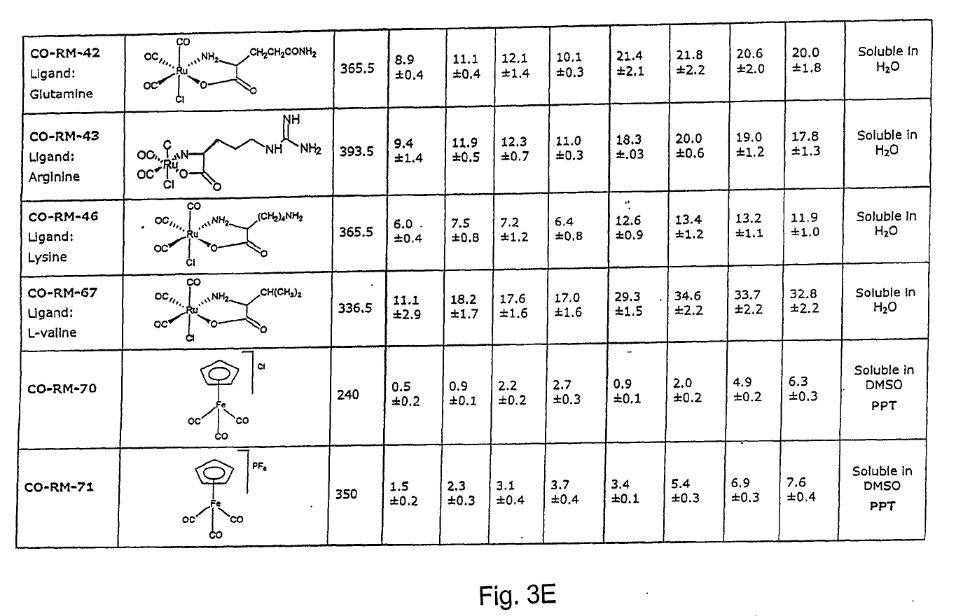

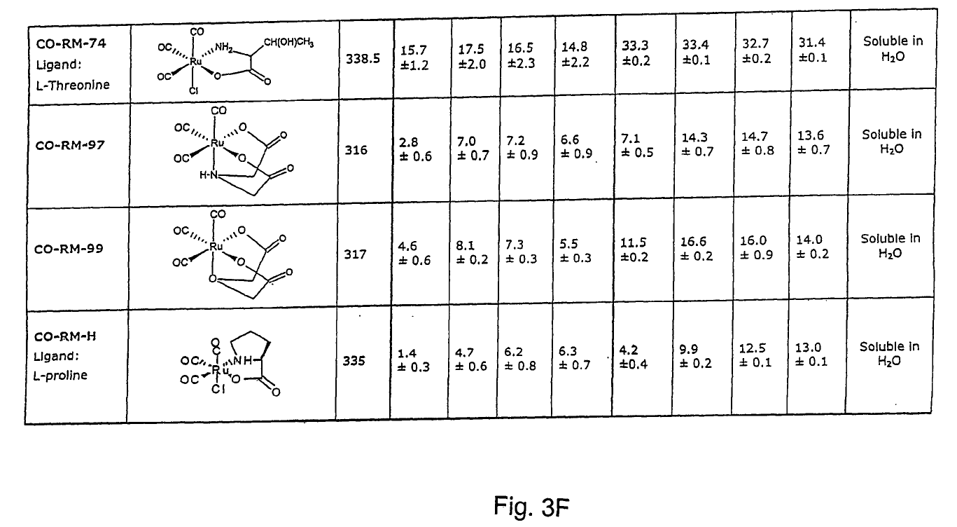

Syntheses Synthetic methods for obtaining the metal carbonyl compounds of FIGS. 3A to 3F were described in W002/092075, the entire content of which is incorporated herein by reference. These compounds are examples of those useful in the present invention. The CO release data in FIGS. 3A to 3F is explained in W002/092075.

Preparation of Ru(CO)₃Cl(NH₂CH₂CO₂) [M_{R} 294.5] Glycine complex. Reference number: CORM-3

[Ru(CO)₃Cl₂]₂ (0.129 g, 0.25 mmol) and glycine (0.039 g, 0.50 mmol) were placed under nitrogen in a round bottomed flask. Methanol (75 cm³) and sodium ethoxide (0.034 g, 0.50 mmol) were added and the reaction stirred for 18 hours. The solvent was then removed under pressure and the yellow residue redissolved in THF, filtered and excess 40-60 light petroleum added. The yellow solution was evaporated down to give a pale yellow solid (0.142 g, 96%). CORM-3 was stored in closed vials at 4 C and used freshly on the day of the experiments.

Alternative, preferred preparation of Ru(CO)₃Cl(NH₂CO₂CO₂) [M_{R}294.6] Glycine complex. Reference number: CORM-3

[Ru(CO)₃Cl₂]₂ (0.129 g, 0.25 mmol) and glycine (0.039g, 0.50 mmol) were placed under nitrogen in a round bottomed flask. Methanol (40 cm³) and sodium methoxide (0.5 M solution in MeOH, 1.00 cm³, 0.50 mmol) were added and the reaction stirred for 18 hours. HC1 (2.0 M solution in diethyl ether) was added in small aliquots until the IR band at 1987 cm⁻¹ in solution IR spectroscopy could no longer be detected. The solvent was then removed under reduced pressure and the yellow residue redissolved in THF, filtered and an excess of 40-60 light petroleum added. The resulting precipitate was isolated by pipetting off the mother liquor and drying under high vacuum. The same work up was repeated for the mother liquor once concentrated. The colour of the product varied between white and pale yellow and was produced in an average yield of 0.133 g, (90%).

### EXAMPLE 1

Preparation of isolated rat aortic rings and experimental protocol

The method for the preparation of isolated aortic rings has been previously described (5; 7). The thoracic aorta was isolated from Sprague-Dawley rats (350-450 g) and flushed with cold Krebs-Henseleit buffer (4° C., pH 7.4) containing (in mM): 118 NaCl, 4.7 KCl, 1.2 KH₂PO₄, 1.2 MgSO₄.7H₂O, 22 NaHCO₃, 11 Glucose, 0.03 K⁺EDTA, 2.5 CaCl₂ and supplemented with 10 AM indomethacin. Each aorta was trimmed of adventitial tissue and ring sections (˜3 mm length) were produced from the mid aortic segment. The rings were then mounted between two stainless steel hooks in 9-ml organ baths containing Krebs-Henseleit buffer which was maintained at 37° C. and continuously gassed with 95% O₂-5% CO₂. One hook was attached to a Grass FT03 isometric force transducer whilst the other was anchored to a sledge for regulation of the resting tension of the aortic ring. The rings were initially equilibrated for 30 min under a resting tension of 2 g which was previously determined to be optimal. Continuous recording of tension was made on a Grass 7D polygraph (Grass Instruments, Quincy, Mass.) in combination with a Biopac MP100 system using AcqKnowledge™ software (Linton Instruments, Norfolk, UK). Before each protocol was carried out, rings were contracted with a standard dose of KCl (100 mM) in order to provide an internal reference and to control for variability in contractile responsiveness between tissues. The relaxation response to CORM-3 (25 μM) in the presence or absence of YC-1 (5 μM final concentration, 30 min pre-incubation) was assessed in aortic rings pre-contracted with phenylephrine (1 Amol/L).

Results

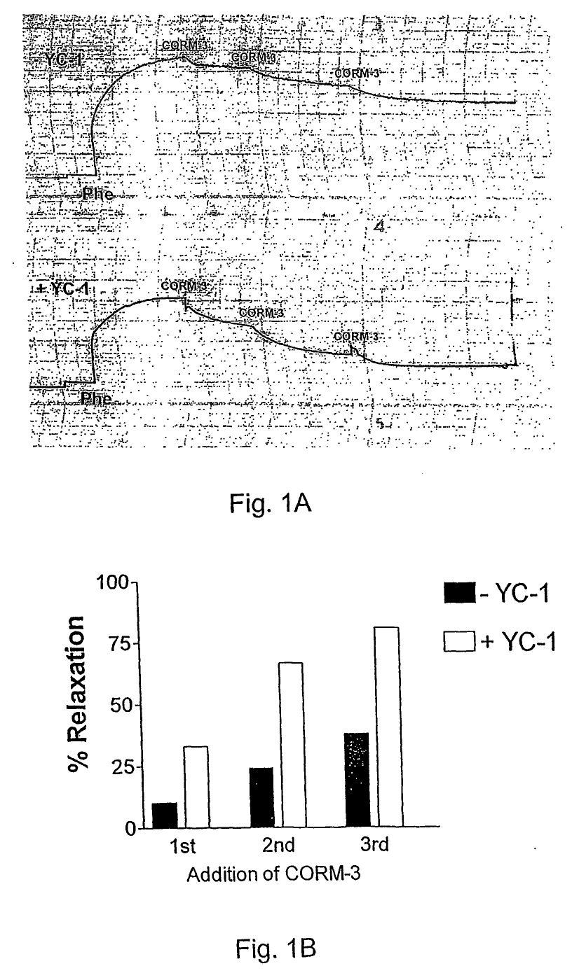

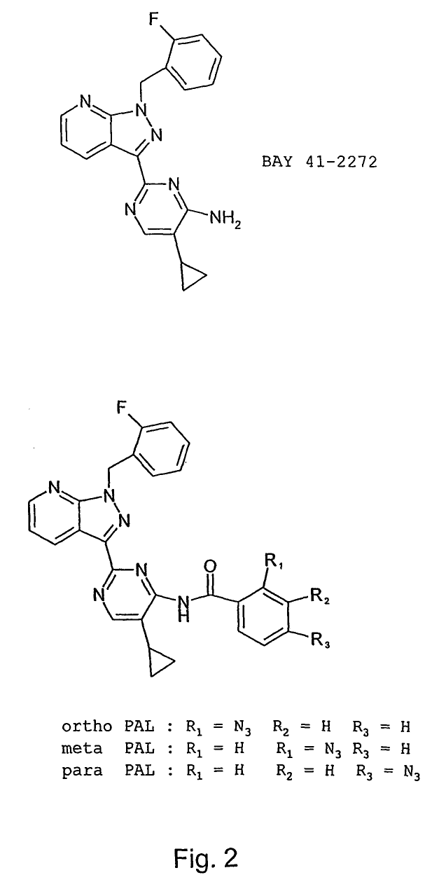

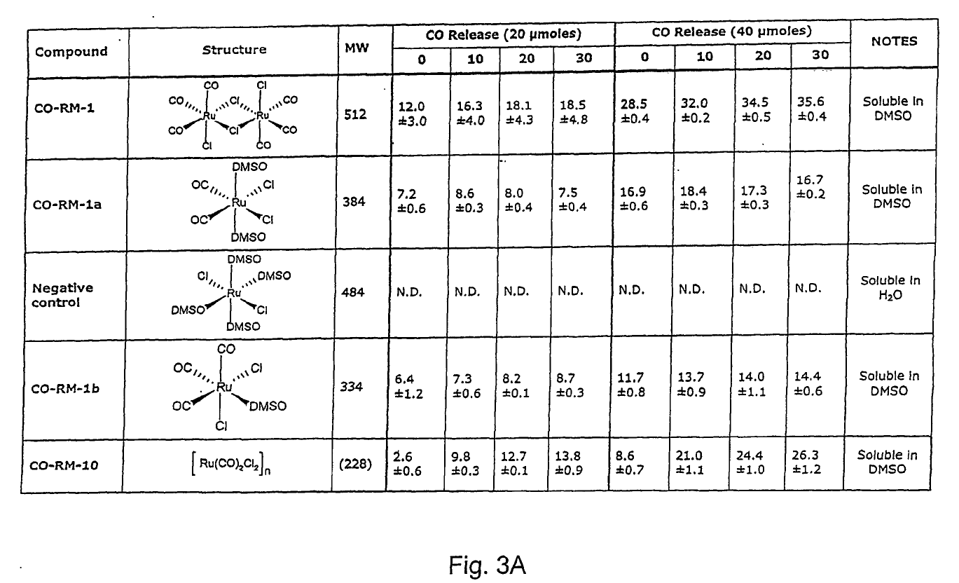

FIG. 1A shows the typical plots of the vascular reactivity to phenylephrine and the vasodilatory effects of CORM-3 alone or in combination with YC-1 in this Example. In the absence of YC-1, three sequential additions of CORM-3 (25 AM each) to the pre-contracted ring elicited vasorelaxation (see top plot). If the relaxation is expressed as a percentage of the maximal phenylephrine-mediated contraction, then we can calculate that CORM-3 produced a 10.3% relaxation after the first addition, 24.1% relaxation after the second addition and 38% after the third addition (FIG. 1B). The presence of YC-1 in the organ bath amplified the observed vasodilatory effect mediated by CORM-3 (see bottom plot, FIG. 1A) and produced a 33% relaxation after the first addition of the CO carrier, 66.6% relaxation after the second addition and 80.9% after the third addition (FIG. 1B). These data indicate that CO released by CORM-3 mediates a vasodilatory effect which can be further enhanced by addition of the sGC activator YC-1. In view of the fact that increased cGMP levels by YC-1 in the presence of CO led to complete inhibition of platelet aggregation (1), the results presented here point to the potential therapeutic use of CORM-3 in combination with YC-1 in those pathophysiological conditions characterized by increased platelet aggregation.

Example 2 and Example 3 are presented here as background, to illustrate the effects of CORM-3 in CO release and vasorelaxation.

### EXAMPLE 2

Conversion of myoglobin (Mb) to carbon monoxide myoglobin (MbCO) by CO gas and CORM-3.

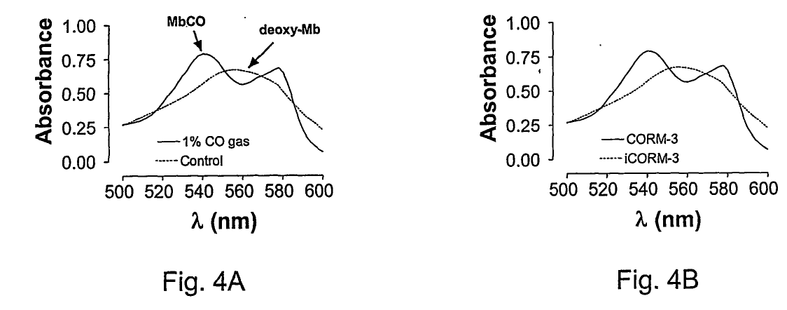

Myoglobin (Mb) in its reduced state displays a characteristic spectrum with a maximal absorption peak at 555 nm (see FIG. 4A, dotted line). When a solution of Mb (50 μM) is bubbled for 1 min with CO gas (1%), a rapid conversion to carbon monoxide myoglobin (MbCO) is observed. As shown in FIG. 4A (solid line), MbCO displays a characteristic spectrum with two maximal absorption peaks at 540 and 576 nm, respectively. This method has been previously developed to monitor and determine the amount of CO released from CO-RMs (7). Indeed, when CORM-3 ([Ru(CO)₃Cl(glycinato)] is first solubilized in water and then added to the Mb solution, formation of MbCO is observed (FIG. 4B, solid line). The amount of MbCO formed is instantaneous and indicates that 1 mole of CO per mole of CORM-3 is promptly released (7). Interestingly, CO is rapidly lost when CORM-3 is left incubating overnight at 37° C. in phosphate buffer at pH=7.4. This step allows the generation of an inactive compound (iCORM-3) that does not convert Mb to MbCO (see FIG. 4B, dotted line) and is used as negative control of CORM-3 when testing for its pharmacological activities.

### EXAMPLE 3

Comparison between CORM-3 and iCORM-3 in their ability to elicit vasorelaxation.

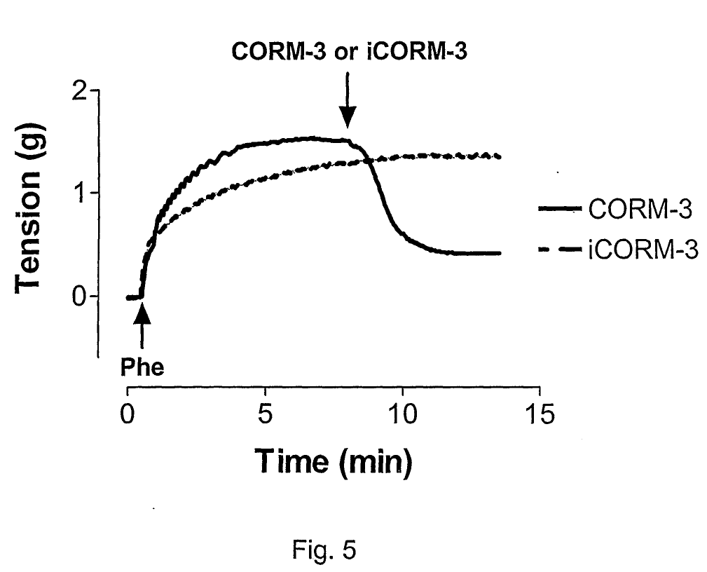

CORM-3 (100 μM) added to isolated aortic rings pre-contracted with phenylephrine (Phe) promoted approximately 54% relaxation within few minutes from addition (See FIG. 5, solid line). In contrast, 100 μM iCORM-3 (which is incapable of releasing CO) did not cause any significant change in vessel tone (see FIG. 5, dotted line). These results indicate that CO liberated from CORM-3 is directly responsible for the observed pharmacological effect.

### EXAMPLES 4A, 4B and 4C

Preparation of isolated rat aortic rings and experimental protocol

The preparation of isolated aortic rings and recording of tension was as in Example 1. Before each protocol was carried out, rings were contracted with a standard dose of KCl (100 mM) in order-to provide an internal reference and to control for variability in contractile responsiveness between tissues. The relaxation response to CORM-3 (25, 50 and 100 μM) in the presence and absence of YC-1 (1 μM final concentration, 30 min pre-incubation) was assessed in aortic rings pre-contracted with phenylephrine (1 μm). CORM-3 was added to the aortic rings as three cumulative additions at 10 minute intervals and the percentage of relaxation produced was recorded after each addition. In another set of experiments, an inhibitor of guanylate cyclase (H-(1,2,4) oxadiazolo (4,3-a) quinoxallin-1-one, here called ODQ, 10 μM) and an inhibitor of ATP-dependent potassium channels (glibenclamide, 10 μM) were tested for their ability to modulate CORM-3 -dependent vasorelaxation. ODQ and glibenclamide were added to the aortic ring preparation prior to CORM-3 addition (15 min and 30 min, respectively). To test the possibility that the nitric oxide (NO) synthase pathway is involved in the vasorelaxing effects mediated by CORM-3, a NO synthase inhibitor (L-nitroarginine methyl esther or L-NAME, 10 μM) was added to the aortic rings 30 min prior to CORM-3 addition. An additional set of experiments was performed in which the vascular endothelium was removed from the aortic tissue prior to CORM-3 addition. As an index of direct guanylate cyclase activation by CORM-3, the levels of cyclic guanosine monophosphate (cGMP) were also measured in freeze-clamped aortic tissue extracts using a commercial ELISA kit (Amersham) as previously described (7). The levels of cGMP in aortic tissue was measured 8 min after each of the three cumulative additions of CORM-3 (100 μM) and compared to the basal levels of cGMP (control, no treatment).

### RESULTS-EXAMPLE 4A

Effects of guanylate cyclase and potassium channel inhibitors on the vasorelaxation mediated by CORM-3.

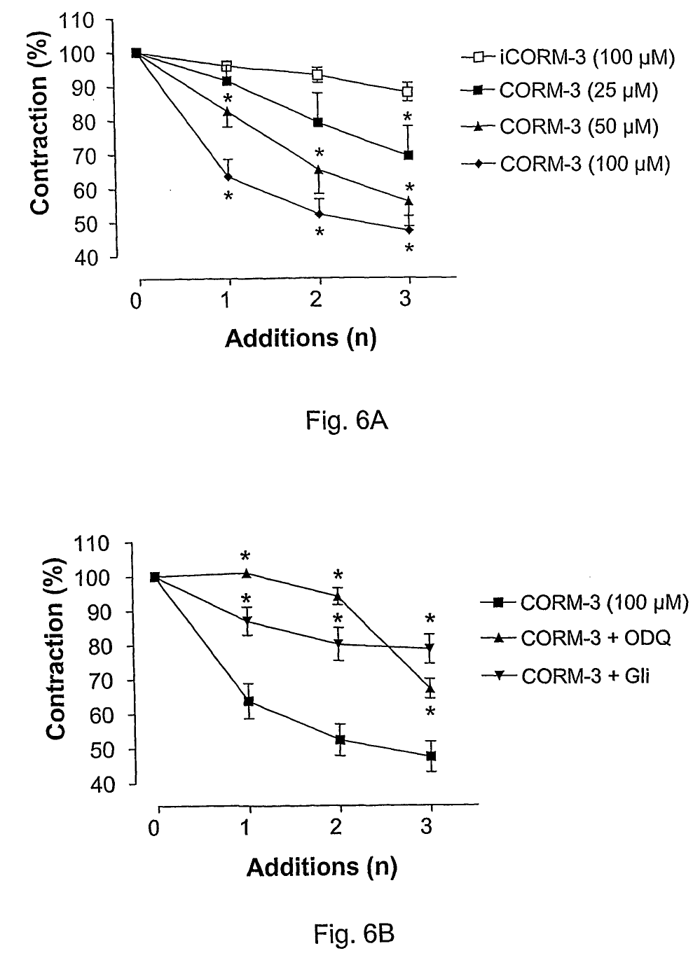

In FIGS. 6, 7 and 8, in the graphs reporting construction, the 100% value at “Addition O” is the value of vasocontractility before addition of CORM-3.

Pre-contracted aortic rings were treated with increasing concentrations of CORM-3 (25, 50 and 100 μM) as described above. Three cumulative additions of CORM-3 were given and the percentage of vasorelaxation was calculated at the end of each addition. As shown in FIG. 6A, CORM-3 caused a significant relaxation in a concentration-dependent manner (reported in the graph as a decrease in contraction). It can be seen from the graph that after treatment with 100 μm CORM-3, the percentage of relaxation elicited by the three cumulative additions were 37.5±5.3%, 48.2±4.4% and 53.9±4.3%, respectively. Addition of iCORM-3 (100 μM), which does not release CO, did not produce any detectable change in vessel contractility. The data are represented as the mean±S.E.M. of 6 independent experiments for each group (*p<0.05 vs. iCORM-3). FIG. 6B shows the effect of ODQ (a guanylate cyclase inhibitor) and glibenclamide (Gli, an inhibitor of ATP-dependent potassium channels) on CORM-3-mediated vasorelaxation. Both inhibitors were very effective in attenuating the relaxation caused by CORM-3. For instance, the 37.5±5.3% relaxation elicited by CORM-3 following the first addition was reduced to 1.0±0.8% and 13.2±4.1% in the presence of ODQ and glibenclamide, respectively. During the first two additions of CORM-3, the inhibition of relaxation was more pronounced with ODQ. The data are represented as the mean±S.E.M. of 6 independent experiments for each group (*p<0.05 vs. CORM- 3).

### RESULTS-EXAMPLE 4B

Effect of YC-1 on CORM-3-mediated vasorelaxation.

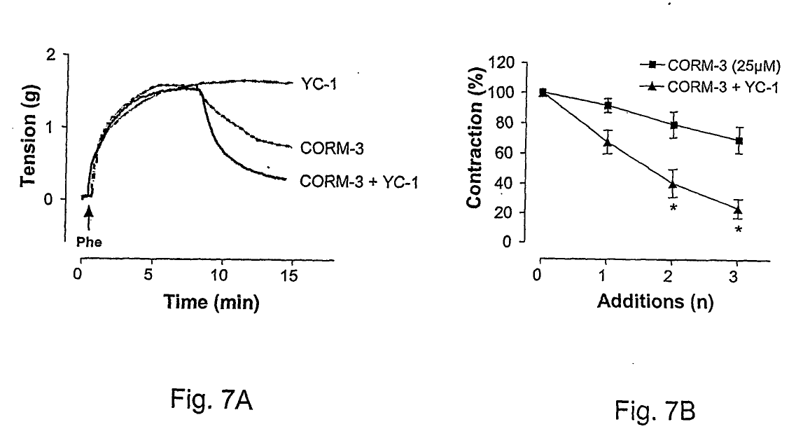

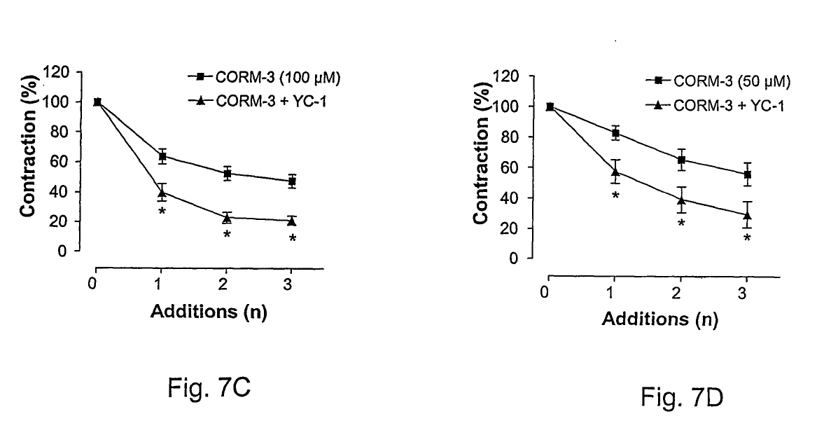

YC-1 sensitizes guanylate cyclase (sGC) to the effect of CO gas as previously reported by Friebe and colleagues (1; 2). To test whether a similar effect could be observed in the presence of CORM-3, YC-1 was added to the aortic ring preparation 30 min prior to the addition of CORM-3. As shown in FIG. 7A, the presence of YC-1 potentiated the relaxation elicited by CORM-3 (note: pre-treatment of YC-1 alone did not cause any significant change in vessel contractility). Specifically, YC-1 significantly amplified the reduction in contractility mediated by CORM-3 at all concentrations used. For instance, after the third addition, 25 μM CORM-3 alone caused approximately 31% relaxation whereas pre-treatment of vessels with YC-1 increased the extent of relaxation to 77% (FIG. 7B). A similar pattern showing a potentiation of relaxation by YC-1 was obtained in experiments using 50 μM (FIG. 7C) and 100 μM (FIG. 7D) CORM-3. These results indicate that CO released by CORM-3 mediates a vasodilatory effect which can be further enhanced by addition of the sGC activator YC-1. The data are represented as the mean±S.E.M. of 6 independent experiments for each group (*p<0.05 vs. CORM-3).

### RESULTS-EXAMPLE 4C

Effects of L-NAME and removal of the endothelium on CORM-3-mediated vasorelaxation.

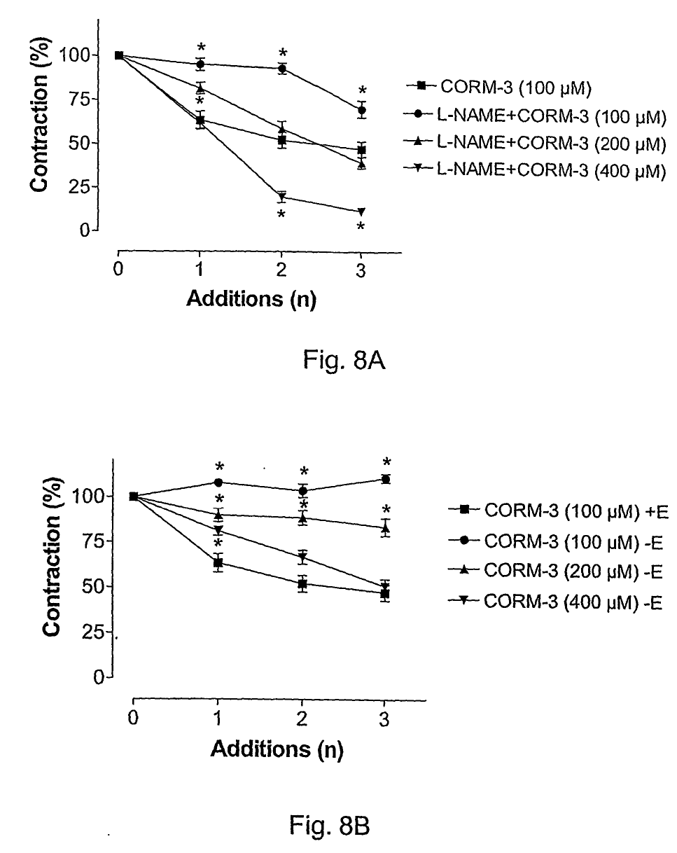

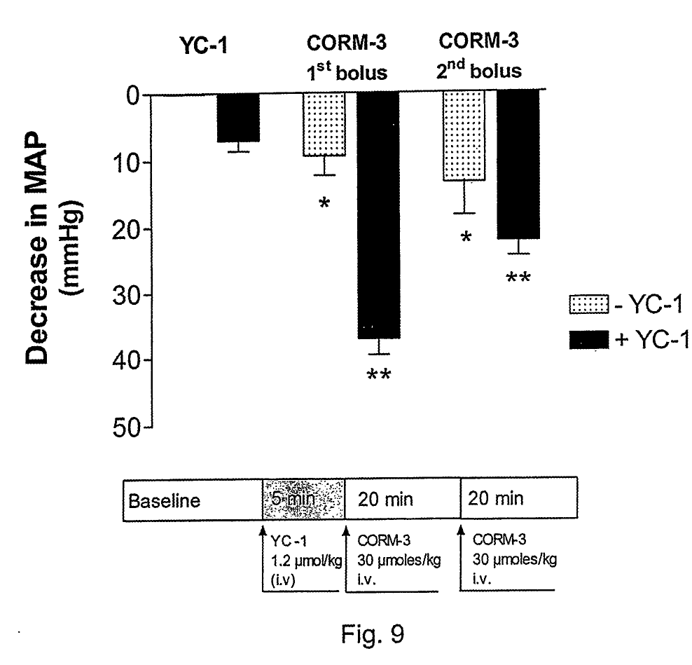

The NO synthase inhibitor, L-NAME, was used to ascertain whether CORM-3 mediates its effects through a mechanism involving the endogenous generation of NO. As shown in FIG. 8A, L-NAME (100 μM) significantly attenuated the relaxation effect elicited by each addition of CORM-3. For instance, following the first addition, CORM-3 caused approximately 37% relaxation but the presence of L-NAME in the organ bath reduced the extent of relaxation to 5%. Interestingly, the effect of L-NAME was reversed by increasing the concentration of CORM-3 (200 and 400 μM). A similar effect was obtained by removing the endothelium from the vessel. As shown in FIG. 8B, aortic rings without the endothelium (indicated in the graph by −E) did not respond to CORM-3 in the same way as intact vessels (+E) unless the concentration of CORM-3 was increased to 400 μM. These results indicate that NO and other factors produced by the endothelium facilitates vasorelaxing capacity of CORM-3 to promote vasorelaxation. The data are represented as the mean±S.E.M. of 6 independent experiments for each group (*p<0.05 vs. CORM-3).

### EXAMPLE 5

Effect of CORM-3 and YC-1 on mean arterial pressure.

Lewis rats (280-350 g) were anaesthetised by intramuscular injection of 1 ml/kg Hypnorm. Specially designed femoral artery and venous catheters were then surgically implanted as previously described (5; 6) and blood pressure monitored continuously using a polygraph recorder. Rats were administered with one bolus of CORM-3 (30 μmoles/kg) and after 20 min with a second bolus. CORM-3 was injected intravenously and the change in mean arterial pressure MAP recorded 20 min after each injection. In the experiments using YC-1, the sGC activator (1.2 μmoles/kg) was administered intravenously 5 min prior to the first injection of CORM-3. The effect of CORM-3 alone or in combination with YC-1 on mean arterial pressure (MAP) in vivo is summarized in FIG. 7. Two bolus of CORM-3 (30 μmoles/kg) were injected intravenously 20 min apart from each other and the change in MAP recorded. In the experiments using YC-1, the sGC activator (1.2 μmoles/kg) was administered intravenously 5 min prior to the first injection of CORM-3. As shown, CORM-3 alone produced a rapid and significant decrease in MAP; specifically, the first injection of CORM-3 produced a decrease in MAP of 9.4±2.8 mmHg whereas the second bolus of CORM-3 resulted in a drop of 13.3±4.9 mmHg. The injection of YC-1 potentiated the hypotensive effects mediated by CORM-3. Administration of YC-1 lead to a decrease in MAP (7.0±1.6 mmHg) after 5 min; the subsequent injections of CORM-3 amplified this effect resulting in a decrease in MAP of 37.0±2.4 and 22.0±2.3 mmHg after the first and second injection, respectively. These results indicate that CO released by CORM-3 mediates an hypotensive effect in vivo which can be further enhanced in combination with the sGC activator YC-1.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

References:

1. Friebe A, Mullershausen F, Smolenski A, Walter U, Schultz G and Koesling D. YC-1 potentiates nitric oxide- and carbon monoxide-induced cyclic GMP effects in human platelets. Mol Pharmacol 54: 962-967, 1998.

2. Friebe A, Schultz G and Koesling D. Sensitizing soluble guanylyl cyclase to become a highly CO-sensitive enzyme. Embo J 15: 6863-6868, 1996.

3. Furchgott R F and Jothianandan D. Endothelium—dependent and—independent vasodilation involving cGMP: relaxation induced by nitric oxide, carbon monoxide and light. Blood Vessels 28: 52-61, 1991.

4. Moncada S, Palmer R M J and Higgs E A. Nitric oxide: physiology, pathophysiology, and pharmacology. Pharmacol Rev 43: 109-142, 1991.

5. Motterlini R, Clark J E, Foresti R, Sarathchandra P, Mann B E and Green C J. Carbon monoxide-releasing molecules: characterization of biochemical and vascular activities. Circ Res 90: E17-E24, 2002.

6. Motterlini R, Gonzales A, Foresti R, Clark J E, Green C J and Winslow R M. Heme oxygenase-1-derived carbon monoxide contributes to the suppression of acute hypertensive responses in vivo. Circ Res 83: 568-577, 1998.

7. Sanmut I A, Foresti R, Clark J E, Exon D J, Vesely M J J, Sarathchandra P, Green C J and Motterlini R. Carbon monoxide is a major contributor to the regulation of vascular tone in aortas expressing high levels of haeme oxygenase-1. Br J Pharmacol 125: 1437-1444, 1998.

8. Stone J R and Marletta M A. Soluble guanylate cyclase from bovine lung: activation with nitric oxide and carbon monoxide and spectral characterization of the ferrous states. Biochemistry 33: 5636-5640, 1994.

9. Becker E M et al. NO-independent regulatory site of direct sGC stimulators like YC-1 and BAY 41-2272. BMC Pharmacology 1: 13, 2001.

1. A pharmaceutical preparation, comprising a metal carbonyl compound or pharmaceutically acceptable salt thereof, a guanylate cyclase stimulant or stabilizer and at least one pharmaceutically acceptable carrier.

2. A pharmaceutical preparation according to claim 1, wherein the metal carbonyl makes available CO suitable for physiological effect, for delivery of carbon monoxide to a physiological target.

3. A pharmaceutical preparation according to claim 2, wherein said metal carbonyl compound makes CO available by at least one of the following means:
1) CO derived by dissociation of the metal carbonyl is present in the composition in dissolved form;
2) on contact with a solvent or ligand the metal carbonyl releases CO;
3) on contact with a tissue, organ or cell the metal carbonyl releases CO;
4) on irradiation the metal carbonyl releases CO.

4. A pharmaceutical preparation according to claim 1, wherein said metal carbonyl compound and said guanylate cyclase stimulant/stabilizer are combined in a single composition.

5. A pharmaceutical preparation according to claim 1, wherein said metal carbonyl compound and said guanylate cyclase stabilizer/stimulant are in separate compositions for administration simultaneously or sequentially.

6. A pharmaceutical preparation according to claim 1 wherein the metal carbonyl compound has the formula M(CO)ₓA_{y} where x is at least one, y is at least one, M is a metal, the or each A is an atom or group bonded to M by an ionic, covalent or coordination bond but is not CO, and in the case where y>1 each A may be the same or different, or a pharmaceutically acceptable salt of such a compound.

7. A pharmaceutical preparation according to claim 6, wherein M is a transition metal.

8. A pharmaceutical preparation according to claim 6 wherein A is selected from neutral or anionic ligands, such as halide, or derived from Lewis bases and having N, P, O, S or C as the coordinating atom(s).

9. A pharmaceutical preparation according to any one of claim 1, wherein the metal carbonyl compound has the formula
M(CO)ₓA_{y}B_{z}
where
M is Fe, Co or Ru,
x is at least one,
y is at least one,
z is zero or at least one,
each A is a ligand other than CO and is monodentate or polydentate with respect to M and is selected from the amino acids
alanine
arginine
asparagine
aspartic acid
cysteine
glutamic acid
glutamine
glycine
histidine
isoleucine
leucine
lysine
methionine
phenylalanine
proline
serine
threonine
tryptophan
tyrosine
valine
[O(CH₂COO)₂]²⁻and
[NH(CH₂COO)₂]²⁻, and
B is optional and is a ligand other than CO.

10. A pharmaceutical preparation according to claim, 1 wherein the guanylate cyclase stimulant/stabilizer is YC-1.

11. A pharmaceutical composition according to claim 1, adapted for delivery by an oral, intravenous, subcutaneous, nasal, inhalatory, intramuscular, intraperitoneal or suppository route.

12. A method of introducing a therapeutic agent to a mammal comprising the step of administering a pharmaceutical preparation according to any one of claim 1.

13. A method of introducing a therapeutic agent to a mammal comprising:
a) administering a metal carbonyl; and
b) administering a guanylate cyclase stimulant or stabiliser.

14. A method according to claim 13, wherein the metal carbonyl makes CO available for physiological effect, for delivery of CO to a physiological target.

15. A method according to claim 14, wherein said metal carbonyl compound makes CO available by at least one of the following means:
1) CO derived by dissociation of the metal carbonyl is present in the composition in dissolved form;
2) on contact with a solvent or ligand the metal carbonyl releases CO;
3) on contact with a tissue, organ or cell the metal carbonyl releases CO;
4) on irradiation the metal carbonyl releases CO.

16-22. (canceled)

23. A method according to claim 15, wherein the metal carbonyl compound and/or the guanylate cyclase stabilizer/stimulant is administered by an oral, intravenous, subcutaneous, nasal, inhalatory, intramuscular, intraperitoneal or suppository route.

24. A method according to claim 12, wherein the metal carbonyl and guanylate cyclase stimulant/stabilizer are administered to an extracorporeal body organ.

25. A method according to claim 12, where the administration is for the stimulation of vasodilation, or for treatment of any of hypertension, such as acute, pulmonary and chronic hypertension, radiation damage, endotoxic shock, inflammation, inflammatory-related diseases such as asthma and rheumatoid arthritis, hyperoxia-induced injury, apoptosis, cancer, transplant rejection, arteriosclerosis, post-ischemic organ damage, myocardial infarction, angina, haemorrhagic shock, sepsis, penile erectile dysfunction, adult respiratory distress syndrome and inhibition of platelet aggregation.

26. A kit for medical treatment comprising a) a metal carbonyl compound; and b) a guanylate cyclase stimulant/stabilizer.

27. A kit according to claim 26, wherein the metal carbonyl is capable of making available CO suitable for physiological effect.

28-35. (canceled)
